(19) United States
(12) Patent Application Publication
(10) Pub. No.: US 2008/0009670 A1
(43) Pub. Date: Jan. 10, 2008

(54) Semiautomatic Apparatus for Sealing and Cutting of Disposable Multilumen Tubing

(75) Inventors: Michael Voloshin, Haifa (IL); Hezi Henkin, Even Yehuda (IL)

Correspondence Address: BRUCE E. LILLING;LILLING & LILLING PLLC, P.O. BOX 560, GOLDEN BRIDGE, NY, 10526, US

(73) Assignee: STRYKER LTD, Haifa (IL)

(21) Appl. No.: 11/774,780

(22) Filed: Jul. 9, 2007

Related U.S. Application Data
(60) Provisional application No. 60/806,880, filed on Jul. 10, 2006.

(51) Int. Cl. A61B 1/00

(52) U.S. Cl. 600/106

(57) ABSTRACT

A semiautomatic device for cutting and sealing of a proximal, exposed portion of a multilumen tubing connected to an operating handle of an endoscopic apparatus is disclosed. The device is suspended on a hanger connected to a vertical post of a stand. The device comprises a working assembly and a support assembly. The device is mounted on the hanger with possibility for rotation of the working assembly and bringing the working head to the exposed portion of the multilumen tubing.

### FIELD OF THE INVENTION

The present invention relates generally to the field of endoscopy and specifically to endoscopic apparatus used for colonoscopic procedures during which a flexible tube is inserted into the rectum and colon for examination of the colon interior for abnormalities. More particularly, the present invention refers to an apparatus and method for cutting and heat-sealing of disposable multilumen tubing, which is used specifically, but not exclusively, with an endoscopic apparatus, having a disposable sleeve covering the flexible tube and which upon inflation enables advancement of the endoscope within the colon.

### BACKGROUND OF THE INVENTION

There are known endoscopes that employ inflatable flexible sleeves for propulsion endoscope within colon.

Voloshin (U.S. Pat. No. 6,485,409) discloses an endoscope, which comprises an endoscopic probe, a bending section for directing the probe within the colon (steering unit), an insertion tube and a flexible covering sleeve or a sheath, which is coupled proximally to the probe. The bending section of the endoscope is located behind the probe. The sleeve is attached to the endoscope in such a manner that its folded section is retained between a cap and an internal spindle, which are located between the insertion tube and the probe head. When inflated, the folded section unfolds over a flange of the internal spindle and an inner portion of the sleeve is pulled in a distal direction.

Eizenfeld (WO/2004/016299; International application PCT/IL03/00661) discloses an endoscope which employs a flexible inflatable sleeve, which before inflation is retained within a dispenser. The dispenser employed in this endoscope has entry and exit ports defining a transit passage through which the endoscope may pass. The dispenser is adapted to capture the sleeve as the endoscope is retracted through the transit passage in a proximal direction. In another embodiment, the dispenser includes an external sleeve fixed to the dispenser and this external sleeve is adapted to be extended from the dispenser when the endoscope is retracted so that the external sleeve covers the flexible sleeve. By virtue of this provision any contamination on the flexible sleeve remains within the external sleeve and does not contact the endoscope or any other objects or areas outside the patient's body. After the endoscope has been removed entirely from the flexible sleeve, the dispenser together with the external sleeve is discarded.

It is mentioned in the above reference that the endoscope is provided with an internal sleeve, which is also known as a multilumen tubing, since it is usually fitted with appropriate passages or lumens as required for irrigation, suction and for passing endoscopic tools therethrough. During the endoscopic procedure the multilumen tubing is in flow communication with the body passage, which is under examination. The multilumen tubing extends along working channel (or so-called guiding channel) provided in the insertion tube.

After the endoscopic procedure is completed, the insertion tube of the endoscope is retracted from the body passage and there is a possibility for contaminating the endoscope's guiding channel by liquid or debris, which enter into the multilumen tubing during the endoscopic procedure. This matter can leak out through the proximal extremity of the multilumen tubing as it is pulled outside through the guiding channel.

It is mentioned in the above reference, that in order to prevent contamination either a tightly-fitting cup is placed over the proximal extremity of the multilumen tubing in order to seal its interior off or the proximal extremity of the multilumen tubing is crimped or heat-sealed.

Unfortunately no details are provided as to how this crimping or hot sealing procedure should be carried out, nor is construction of a device, which should be used for this purpose, disclosed.

Takahashi (U.S. Pat. No. 5,050,585) describes a sheathed endoscope and mentions that, when the multilumen tube is drawn out of the guide channel, contamination of the interior of the guide channel is possible by that mucus left inside the multilumen tubing. To prevent this possibility fusion, cutting of the multilumen tubing is suggested at a position located at its proximal end. The cut portion of the multilumen tubing is fusion-bonded by the heat applied during the fusion cutting process and the open end of the multilumen tubing is thereby closed.

Unfortunately there are no details provided as to construction of a device, which should be used for this purpose.

There exist also references, which describe various devices for sealing and cutting of plastic tubing. Among such references one can mention Selvin (U.S. Pat. No. 2,647,557), EP 507285, DE 40414037, Scherr (U.S. Pat. No. 4,529,858), Hlavinka (U.S. Pat. No. 5,520,218), and Shaposka (U.S. Pat. No. 4,897,138). Unfortunately none of these devices, however, describes a device, which would be dedicated for cutting and sealing of the multilumen tubing of an endoscope.

Bar-Or (WO/2005/110185; International application PCT/IL05/000426), which description is incorporated herein by reference, discloses a device for cutting and sealing of a disposable multilumen tubing of a colonoscope. This device comprises a first jaw and juxtaposed therewith a second jaw. The jaws are provided with respective contact surfaces, which approach the multilument tubing during the cutting and sealing. The device further comprises biasing member exerting pressure on at least one of the contact surfaces to bring them together and a source of energy suitable for heating of at least one of the contact surfaces. In practice the device is configured as a pistol comprising a trigger member for displacing the first jaw with respect to the second jaw against the force of the biasing member. In an embodiment the device is configured as a pliers. The device is designed in such a manner that a nurse or an operator should manually operate the device irrespective whether it is configured as a pistol or pliers.

The disadvantage of this prior art device is associated with its inconvenience in operation. Furthermore, due to its manual operation this device does not ensure repeatability of the operation since location of the seam left in the end of the procedure may vary by operator. This is especially probable if an unskilled operator uses the device.

The lack of repeatability might result in defective seams and insufficient reliability of the sealing. It should be pointed out that this disadvantage becomes intolerable with endoscopes having disposable protective sleeves, since, if spreading of contamination from the proximal end of the multilumen tubing would not be prevented, the use of protection sleeves becomes pointless.

### SUMMARY OF THE INVENTION

The object of the present invention is to provide a new and improved apparatus and method for cutting and sealing of the multilumen tubing of an endooscope.

The further object of the invention is to provide a new and improved apparatus and method for cutting and sealing of the multilument tubing, which is convenient in operation, which does not depend on the operator's experience, which ensures repeatability of results and which reliably prevents the spread of contamination originating from the disposable multilumen tubing used with the endoscope.

For a better understanding of the present invention as well of its benefits and advantages, reference will now be made to the following description of its embodiments taken in combination with the accompanying drawings.

### BRIEF DESCRIPTION OF THE DRAWINGS

FIG. 1 depicts a general view of an endoscopic apparatus, preferably a colonoscopic apparatus, and its main components

FIG. 2 is a partial isometric view of an exposed portion of the multilument tubing located between the entry port at the operating handle and the connector.

FIG. 3 is a general view of the sealing and cutting apparatus of the invention suspended on a stand.

FIG. 4 is a top view of the sealing and cutting apparatus suspended on a stand.

FIG. 5 is a general isometric view of the sealing and cutting apparatus secured on a hanger.

FIG. 6 is a general view of the sealing and cutting apparatus and its main components.

FIG. 7 is an isometric view of a support bracket.

FIG. 8 is a side isometric view of the sealing and cutting apparatus showing how its rotational axis is inclined at a first inclination angle.

FIG. 9 is a frontal isometric view of the sealing and cutting apparatus showing how its rotational axis is inclined at a second inclination angle.

FIG. 10 is a general isometric view of a support arm carrying displacement mechanism used in the sealing and cutting apparatus of the invention.

FIG. 10A is a cross-sectional top view of the support arm depicted in FIG. 10.

FIG. 11 is a general isometric view of a driving portion of the displacement mechanism.

FIG. 12 is an isometric top view of a working head of the working assembly.

FIG. 13 is an isometric view showing the sealing and cutting apparatus in an initial position.

FIG. 14 is an isometric view showing the sealing and cutting apparatus in a sealing position.

FIG. 15 is an isometric view showing the sealing and cutting apparatus in a cutting position.

FIGS. 16 and 17 are enlarged isometric views showing how the exposed region of the multilumen tubing is being sealed and cut.

FIGS. 18 and 19 show an embodiment of the sealing and cutting apparatus.

### DETAILED DESCRIPTION OF THE INVENTION

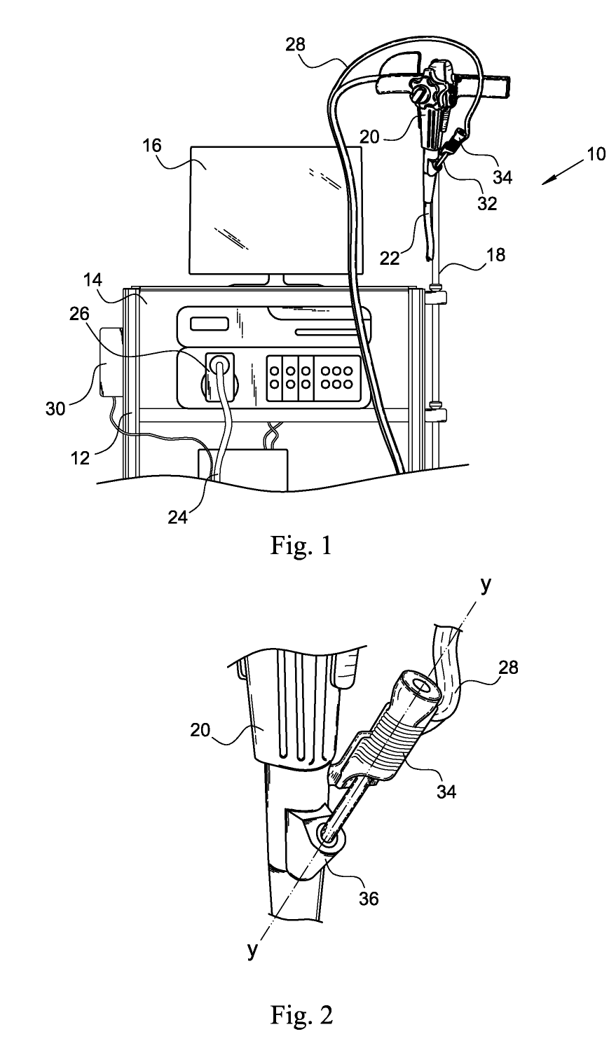

With reference to FIG. 1 an endoscopic apparatus, preferably a colonoscopic apparatus 10, is shown with its following main components. The apparatus comprises accommodated in a cart 12 a system control unit 14 and a monitor 16. The apparatus further comprises a stand 18 with suspended thereon an operating handle 20, connected to an insertion tube 22. The insertion tube is provided at a distal end thereof with an optical head (not shown), which allows visualizing on the monitor the interior of a body passage under investigation. The operating handle is connected with the control unit via an umbilical cord 24 plugged into the control unit through a plug connector 26. There are also provided supply tubes 28 to deliver a fluid medium to the insertion tube. The fluid medium comprises compressed air and water, which is supplied from a dedicated sources provided in the control unit. The source of water can be a flask 30, secured at a side of the control unit. Water is supplied under pressure into the body passage for irrigation. Water and air are delivered to the handle through supply tubes 28, which are attachable to the umbilical duct.

One should bear also in mind that within the insertion tube are provided various devices, which are necessary for proper functioning of the endoscope. These devices are known per se. Among such devices one can mention vertebrae and strings, which can be manipulated by the handle, and a disposable multilumen tubing 32 having appropriate lumens for supplying the fluid medium. The multilumen tubing also is provided with a dedicated passage for introducing surgical instruments into the colon as might be required during the endoscopic procedure.

The multulumen tubing extends through the insertion tube and through the handle to a connector means 34, which provides flow communication between lumens of the multilumen tubing and the supply tubes.

The multilumen tubing enters the insertion tube through a port 36 and it is seen in FIG. 2 that a portion of the multilumen tubing is exposed between the connector and the port. This exposed portion is used for sealing and cutting. When the operating handle is suspended on the stand it is slanted, such that the exposed portion of the multilumen tubing is directed along an axis Y-Y, which is inclined with respect to the stand 18 at an angle γ of about 30-40 degrees.

Description of the connector, of the multilumen tubing and of the apparatus for sealing and cutting of the multilumen tubing can be found in Bar-Or (WO/2005/110204; International patent application PCT/IL05/000425) and Bar-Or (WO/2005/110185; International patent application PCT/IL05/000426), which disclosures are incorporated herein by reference.

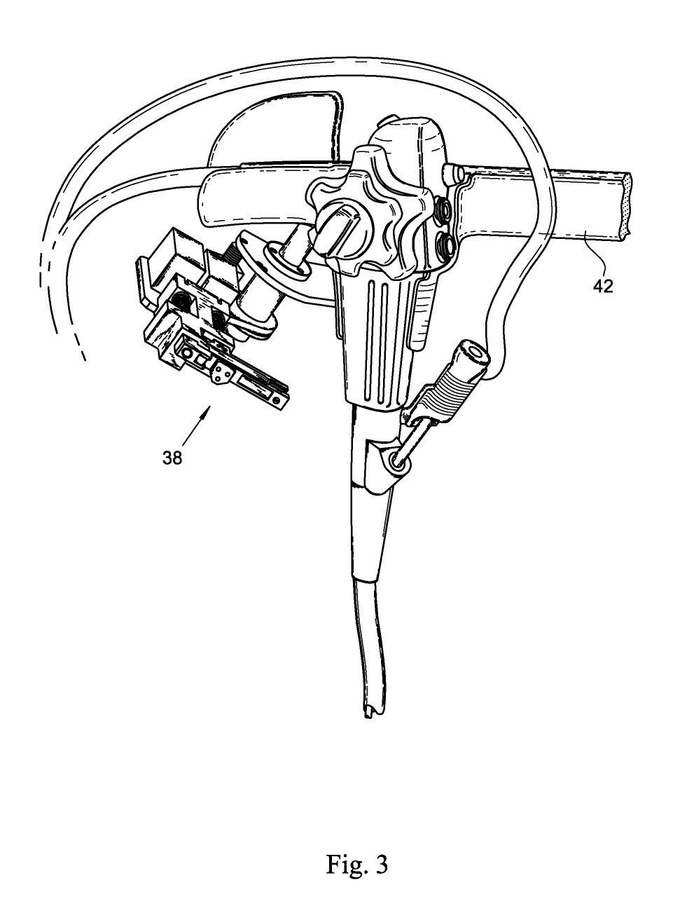

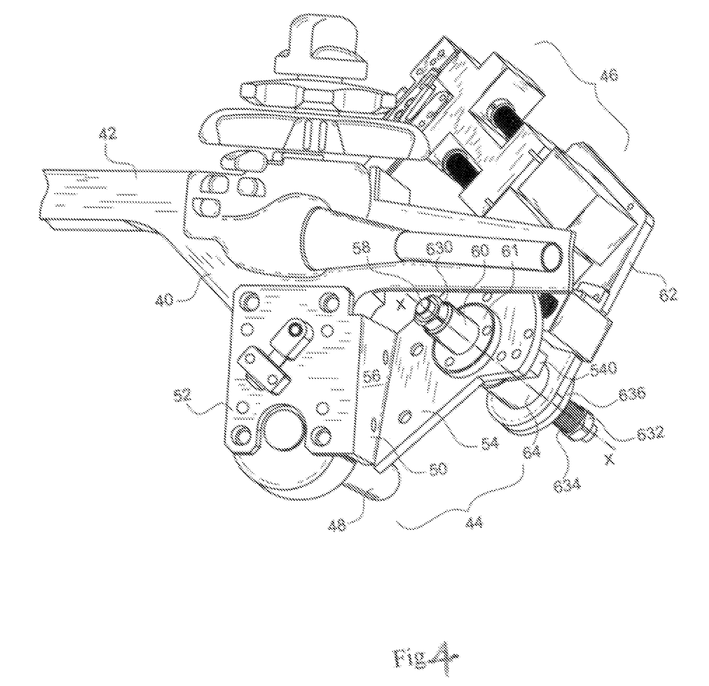

In FIGS. 3, 4 is shown an semiautomatic apparatus 38 for sealing and cutting in accordance with the present invention. The apparatus is secured on a rear portion 40 of a hanger 42 of the stand 18. The apparatus comprises a support assembly 44 and a working assembly 46. The idea of the present invention lies in that the apparatus as mounted on the stand and the working assembly is rotatable about a rotation axis X-X inclined with respect to a vertical post 48 of the stand. By virtue of this provision, the working assembly is allowed to rotate anticlockwise and clockwise. When it is rotated anticlockwise, the working assembly approaches the exposed portion of the multilumen tubing so as to perform sealing and cutting. When it is rotated clockwise, the working assembly can be removed from the exposed portion of the multilumen tubing. By virtue of the inclination, the working assembly is rotatable by gravitation from an initial position remote from the multilumen tubing to a working position proximate to the multilumen tubing. Since the bringing of the working assembly from the initial position to the working position can be effected without applying external force, while its return to the initial position can be carried out forcibly, the apparatus of the present invention is called semiautomatic. It can be readily appreciated that in the apparatus of the present invention, when the rotating working portion returns into the working position, it approaches the same location of the exposed portion of the multilumen tubing. By virtue of this provision, repeatability of the sealing and cutting action is achieved.

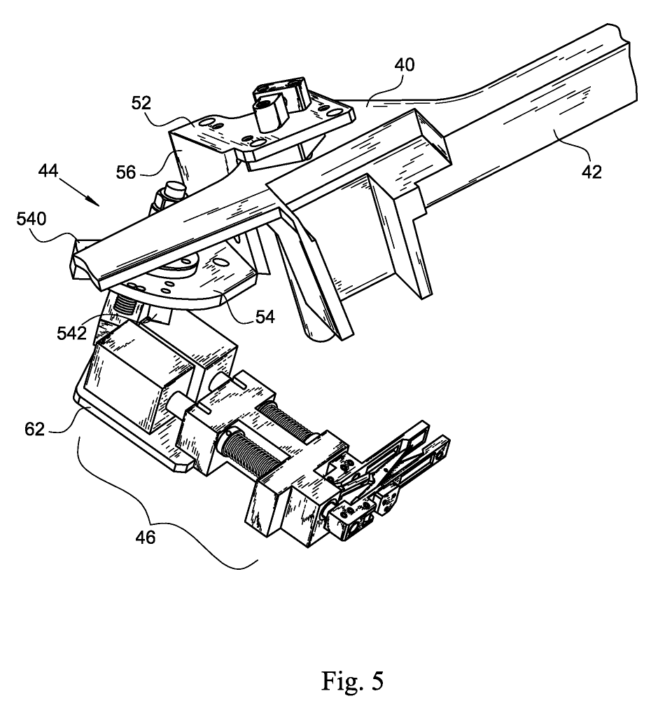

With reference to FIGS. 4, 5 6 and 7 construction of the apparatus and some of its main components would be now explained.

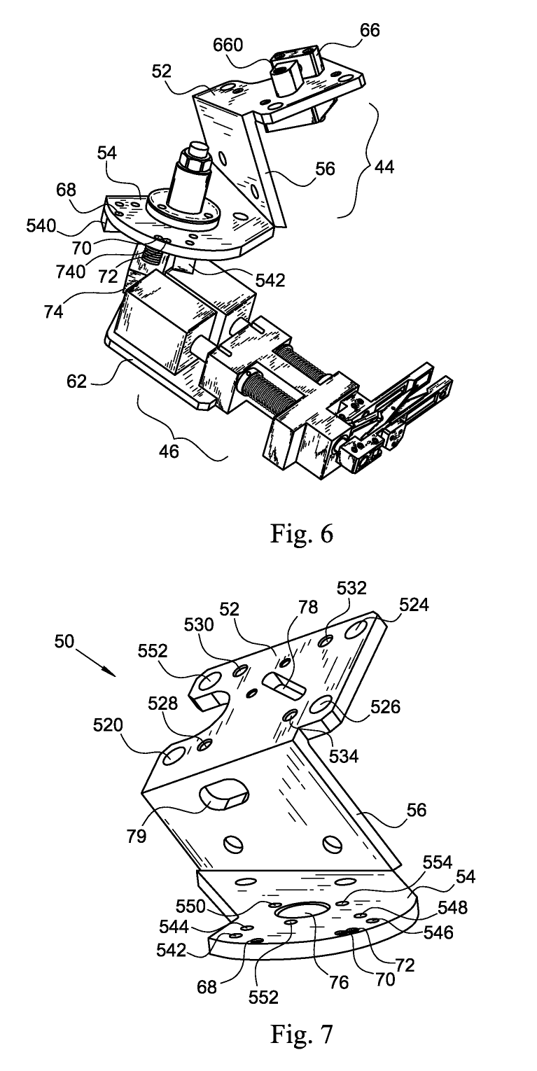

A support assembly secures the apparatus on a rear portion 40 of the hanger 42. The support assembly comprises a Z-like bracket 50 with a flat upper portion 52 embracing the hanger and with a flat lower portion 54. The upper and the lower portion of the bracket are not parallel and an intermediate, vertical portion 56 connects them. Still further elements of the support assembly are a steady shaft 58, a sleeve 60 with a flange 61, a support arm 62 with a sleeve 64, a couple of securing nuts 630, 632, a spring 634 and a lower flange 636. The sleeve 60, the flange 61 and the nut 630 rigidly connect the shaft to the lower portion 54 of the bracket, such that the shaft is steady and does not rotate. The shaft has longitudinal axis X-X, which is inclined with respect to the post 48. The lower flange 636 is pressed by spring 634 and nut 632 to the support arm 62. Since the inner diameter of the sleeve 64 slightly exceeds the outside diameter of the shaft, the support arm is allowed to rotate with respect to the shaft about the axis X-X. The working assembly is carried by the support arm and is rotatable together therewith. Secured on the lower portion 54 of the bracket two stoppers 540, 542 are provided (see shown in FIGS. 5 and 6), which limit rotation movement of the support arm in the clockwise and anticlockwise direction respectively.

FIG. 5 shows the apparatus of the invention secured on the hanger.

FIG. 6 shows the apparatus of the invention including the support assembly and the working assembly. On the upper portion 52 of the bracket are seen components 66, 660 of an adjustment mechanism, which allow small orthogonal displacements of the upper portion with respect to the hanger. By virtue of this provision it is possible to adjust location of the bracket and thus to adjust location of the working assembly 46 with respect to the exposed portion of the multilumen tubing.

The lower portion of the bracket is provided with apertures 68, 70, 72 and the working assembly is provided with a solenoid 74, having a working pin (not seen). The solenoid is mounted on the support arm beneath the lower portion of the bracket and it is normally open, i.e. when it is not energized, the working pin is biased by a spring 740 to protrude from the solenoid. When the pin enters into an aperture, it secures the support arm and the working assembly in a certain discrete position with respect to the stand. When the solenoid is energized, it retracts the working pin from the aperture against the spring. So, for example, when the support arm has been forcibly rotated clockwise and the pin enters the aperture 68, the working assembly is secured in its initial position, i.e. remote from the exposed portion of the multilumen tubing. When the pin is retracted by the solenoid from the aperture 68, the support arm can freely rotate anticlockwise until the working pin enters the aperture 70. Now the working assembly is secured in the first working position, in which it can seal the multilumen tubing. After completing the sealing, the solenoid again retracts the pin and the support arm is free to rotate further in the anticlockwise direction until the pin enters the aperture 72. Now the working assembly is secured in the second working position, in which it can cut the multilumen tubing. After the cutting is completed the solenoid retracts the pin from the aperture 72 and the support arm can be forcibly returned again to the initial position. In the further description solenoid 74 will be referred-to as position solenoid.

The lower flange 636 is pressed by nut 632 to the support arm and this creates friction, which is necessary for controllable rotation movement of the support arm.

In FIG. 7 is shown bracket 50 with its upper, lower and intermediate portions 52, 54, 56. It is seen also that the lower portion is provided with an opening 76 for entering the shaft and that the upper and the intermediate portion is provided with corresponding adjustment slits 78, 79 as required for the adjustment mechanism. The rest of the openings seen in FIG. 7 are as follows: openings 520, 522, 524, 526 are for screws, which secure the upper portion of the bracket on the hanger; openings 528, 530, 532, 534 are for lift-up screws, which allow adjustment of the vertical position of the upper portion with respect to the hanger; openings 542, 544, 546, 548 are for screws, which secure stoppers 540, 542 to the lower portion of the bracket; openings 550, 552, 554 are for screws, which secure flange 60 on the lower portion of the bracket.

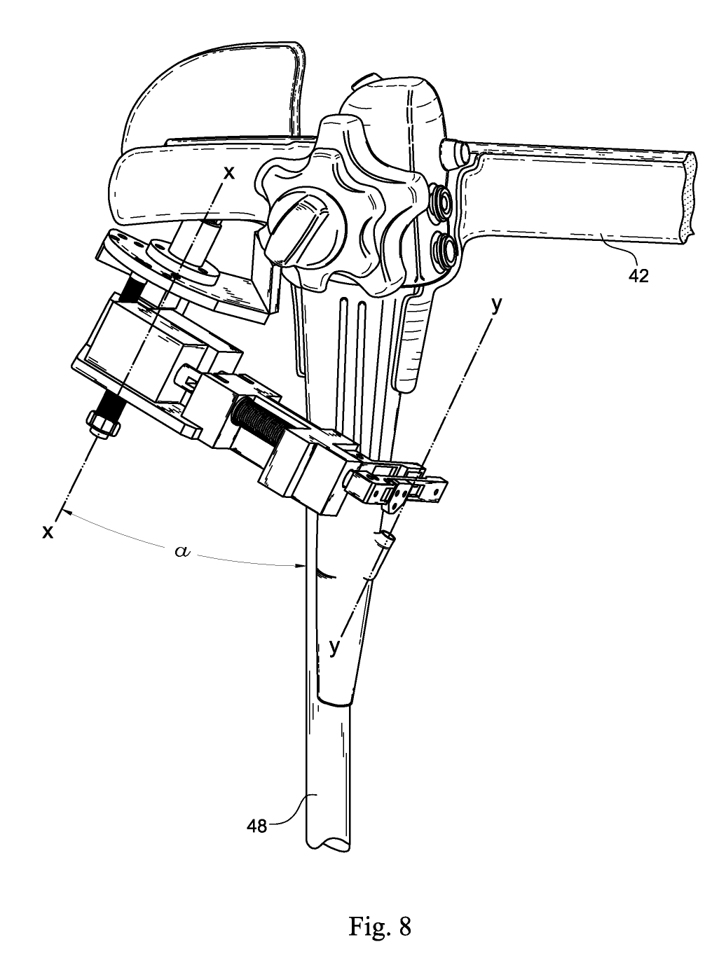

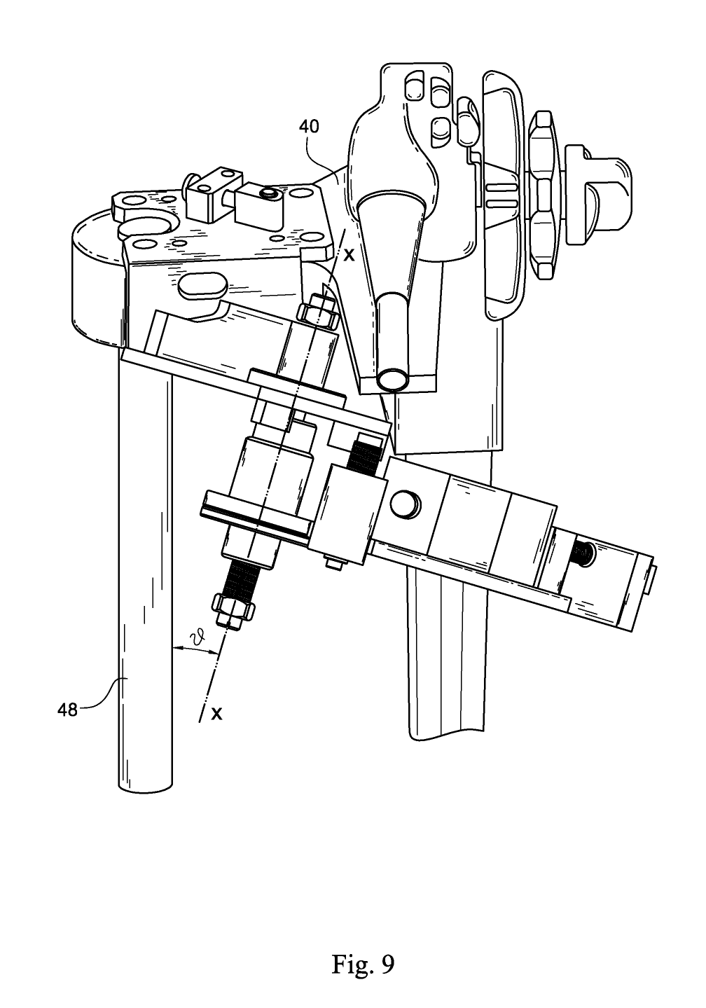

In FIGS. 8 and 9 it is shown how longitudinal axis X-X is inclined with respect to the vertical post of the stand at a first inclination angle α and a second inclination angle β. The first inclination angle a is seen on the frontal view of the apparatus and the second inclination angle β is seen on the side view of the apparatus. The inclination angles are selected to allow easy rotational displacement of the support arm with the carried thereby working assembly. In practice it is advantageous if the longitudinal axis X-X is parallel to the longitudinal axis Y-Y of the exposed portion of the multilumen tubing as shown in FIG. 8. By virtue of this provision the support assembly might have a very simple design, which allows easy approachment of the exposed portion of the multilumen tubing at the same location. In practice the first inclination angle is 30-40 degrees and the second inclination angle is 20-30 degrees.

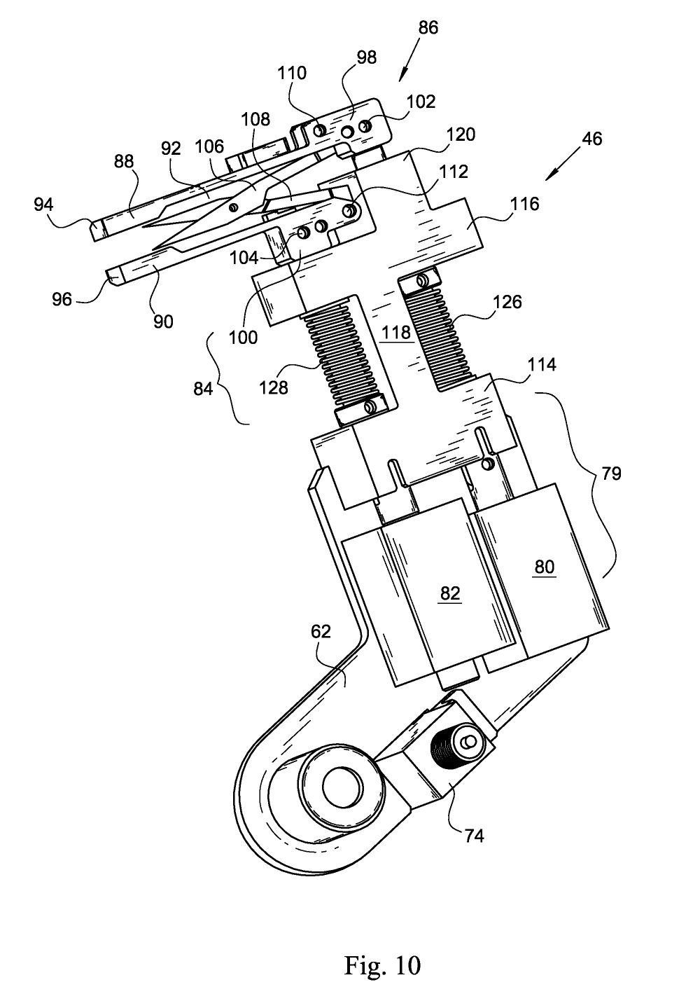

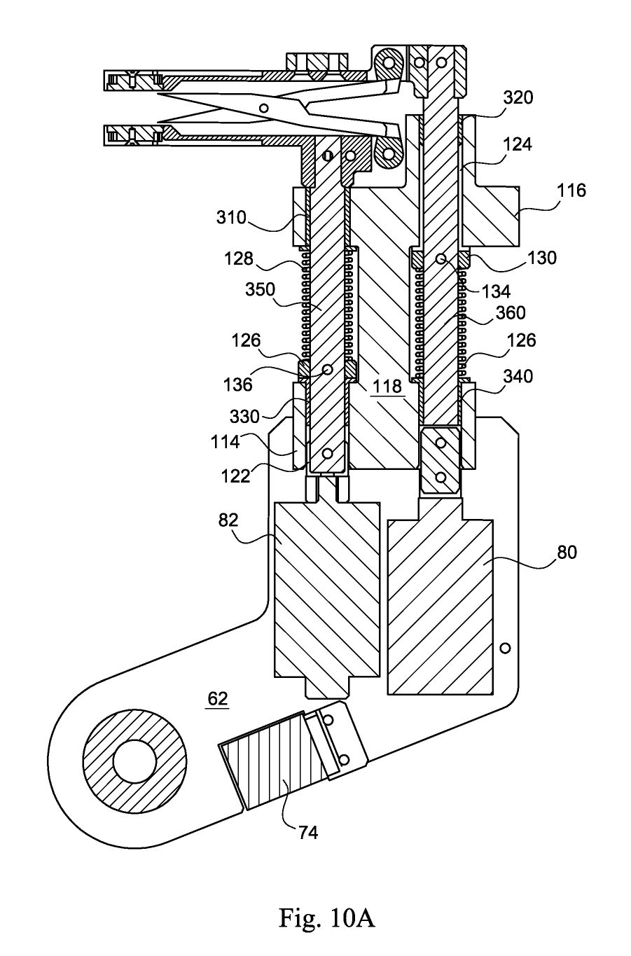

In FIG. 10 is shown the support arm and an embodiment of the working assembly. In this embodiment the working assembly comprises the following main components: a driving portion 79 with two solenoids 80, 82, a guiding portion 84 and a working head 86 with two opposite sealing jaws 88, 90 and with scissors 92. The solenoids are provided with working pins, which can be protracted or retracted. The working pins are connected to traction rods (not shown) by connecting pins, which enter bores made in the working pins. In the further description solenoids 80, 82 will be referred-to as displacement solenoids.

The sealing jaws are provided with heating ends 94, 96 and with respective opposite mounting ends 98, 100. Connecting pins 102, 104 connect the mounting ends to the traction rods. The arrangement being such that the sealing jaws can be brought together or removed from each other when their mounting ends are pushed or pulled by the traction rods. Therefore, if a portion of the multilumen tubing is placed between the heating ends, it can be heat-sealed when the jaws are brought together.

It also seen that the scissors are connected by their ends 106, 108 to the mounting ends of the jaws by respective pins 110, 112. By virtue of this provision the scissors can be closed or opened depending whether the jaws are brought together or removed from each other. By closing the scissors the previously heat-sealed portion of the multilumen tubing can be now cut. The cutting step is carried out when the working assembly has been brought in the second working position as explained above.

Referring to FIG. 10, 10A and 11 the guiding portion comprises a housing provided with a rear transverse portion 114, a forward transverse portion 116 and an intermediate longitudinal portion 118. The forward transverse portion has a protruding region 120 at one side thereof. Within the rear and forward portion are made two parallel through going bores 122, 124, through which the traction rods of the displacement solenoids can pass.

Referring now to FIG. 10A traction rods 350, 360 are seen passing through respective bores 122, 124 made in the guiding portion. Between the guiding portion and the rods there are provided bushings 310, 320, 330 and 340. These bushings are made of plastic material and they function as sliding bearings enabling easy displacement of the traction rods along the guiding portion. Since the bushings are made of electrically insulating plastic they also function as electrical insulators between the rods and the guiding portions. The significance of this provision will be explained further.

On the guiding portion there are also located two biasing elements, which are mounted between the rear and forward portion of the housing. The biasing elements comprise coil springs 126, 128, which are secured at one end thereof on the respective rods by washers 130, 132 and connecting pins 134, 136. The springs are arranged in such a manner that the spring 126 is connected to the rod of solenoid 80 at the forward transverse portion 116, while the spring 128 is connected to the rod of solenoid 82 at the rear transverse portion 114. By virtue of this provision spring 126 urges its rod to be retracted, while spring 128 urges its rod to be protracted. In other words the spring 126 resists to pulling the rod of the displacement solenoid 80, while the spring 128 resists to pushing the rod of the displacement solenoid 82. If the solenoids are not energized, the biasing elements bias the rods in the opposite direction such that the jaws remain brought together. In other words the jaws are in normally closed position. When the multilumen tubing should be placed between the sealing jaws, a room between the heating ends should be provided. To achieve this, one should overcome resistance of the biasing elements and forcibly remove one sealing jaws from another. In order to enable placement of the multilumen tubing between the heating ends, the displacement solenoids should be energized. The displacement solenoids are mounted on the support arm oppositely in the sense that when solenoid 80 is energized it pulls its rod against spring 126 and, when solenoid 82 is energized, it pushes its rod against spring 128. When both displacement solenoids are activated, the sealing jaws would be removed from each other. When the solenoids are not activated, the springs always return the jaws in the position in which the jaws are brought together. It is not shown in the figures, but should be appreciated, that appropriate source of power, wiring and control knobs are provided for energizing the all solenoids, including position solenoid and displacement solenoids.

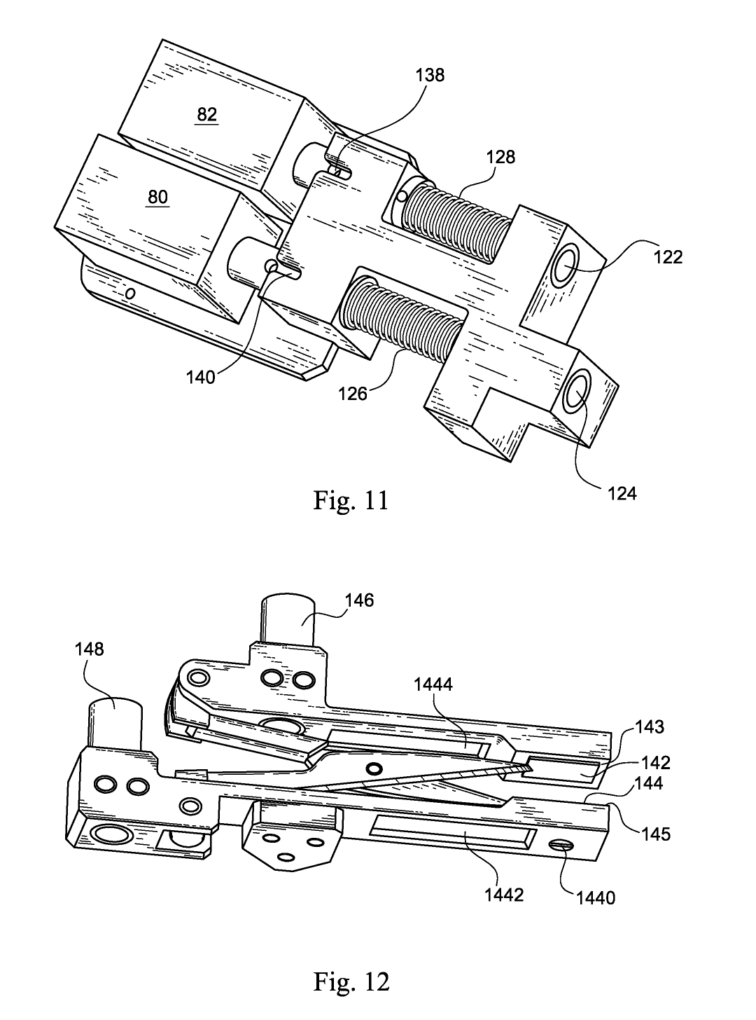

In FIG. 11 is seen the driving and the guiding portion with displacement solenoids, connecting pins, housing, and springs. Elongated slots 138, 140 are made in the rear portion of the housing to allow displacement of the solenoid pins and of the traction rods together with the respective connecting pins. It is seen that the forward portion of the housing has two open ends of the through going bores 122, 124 in which traction rods are displaceable. It should be appreciated that since the pins are allowed to displace within the slots only linearly, the traction rods are prevented from rotation and so the jaws of the working head.

In FIG. 12 is shown a working head with heating jaws and scissors. The heating jaws are provided with contact surfaces 142, 144, which are made of electrically conductive material. The contact surfaces rest on respective bases 143, 145, which are made of a material, which provides electrical and thermal insulation between the jaws and the contact surfaces. The insulating bases are secured at the heating ends of the jaws by fastening screws. One of these screws is seen and it is designated by reference numeral 1440. The contact surfaces are electrically connected to a source of electrical power by appropriate wires (not shown), which are connectable to the jaws. Therefore, when electric current is passed through the contact surfaces, they function as electrical resistance-heating elements capable of transferring heat to any object, which is brought in physical contact with it. In a situation when the surfaces approach each other, the heat passes from one surface to the other surface by conductivity.

The jaws are connected to the traction rods, which are electrically insulated from the rest of the working assembly by bushings 310, 320, 330, 340.

It is not shown, but should be appreciated, that a thermocouple might be arranged on at least on one of the contact surfaces to measure the temperature during the sealing.

The output signal form the thermocouple can be used for automatic control of the electrical power source.

The jaws are provided with lateral windows 1442, 1444, through which the wires for supply of the electrical current and the wiring of the thermocouple can pass.

Secured to one of the jaws by a bracket a micro switch is provided. Situated between the jaws a mechanical stopper is provided (not seen). The micro switch switches the displacement solenoids off as soon as the jaws are brought together and the micro switch approaches the stopper. By virtue of this provision the sealing time can be controlled.

Ports 146, 148 are seen for receiving traction rods and for connecting respective traction rods 350, 360 to the jaws.

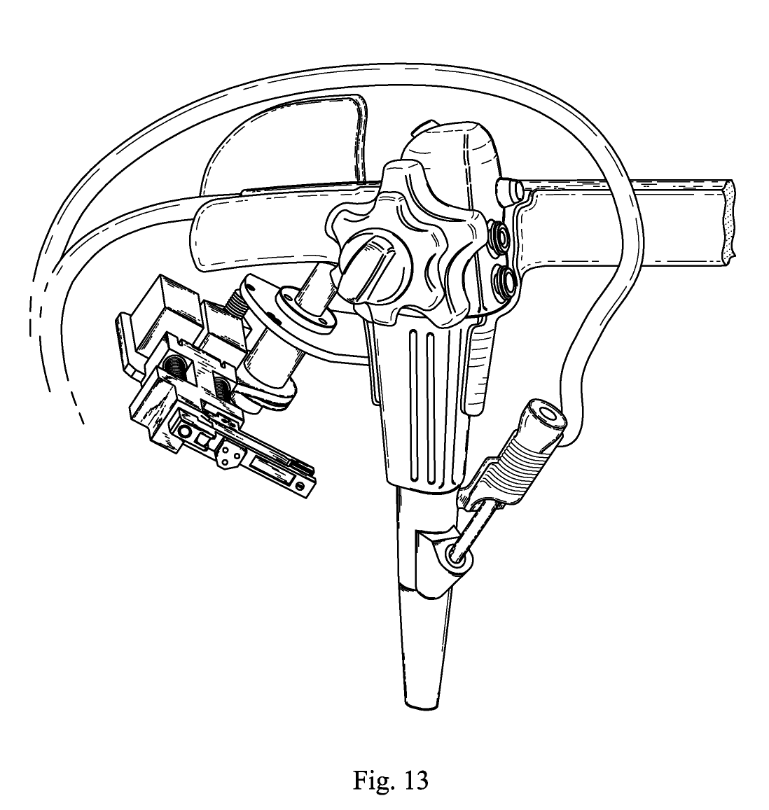

In FIG. 13 is shown the situation when the working assembly is in its initial position.

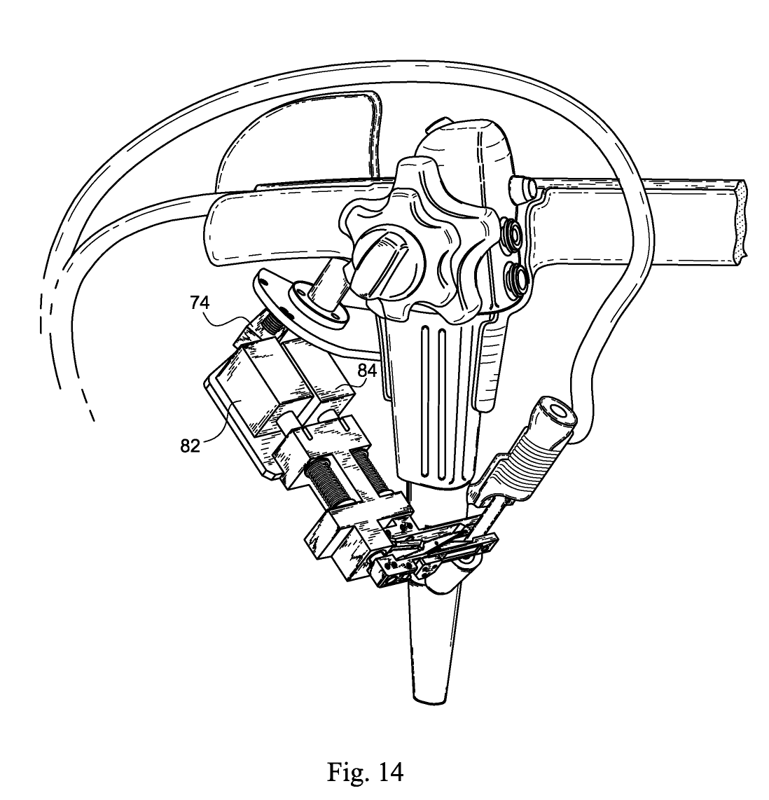

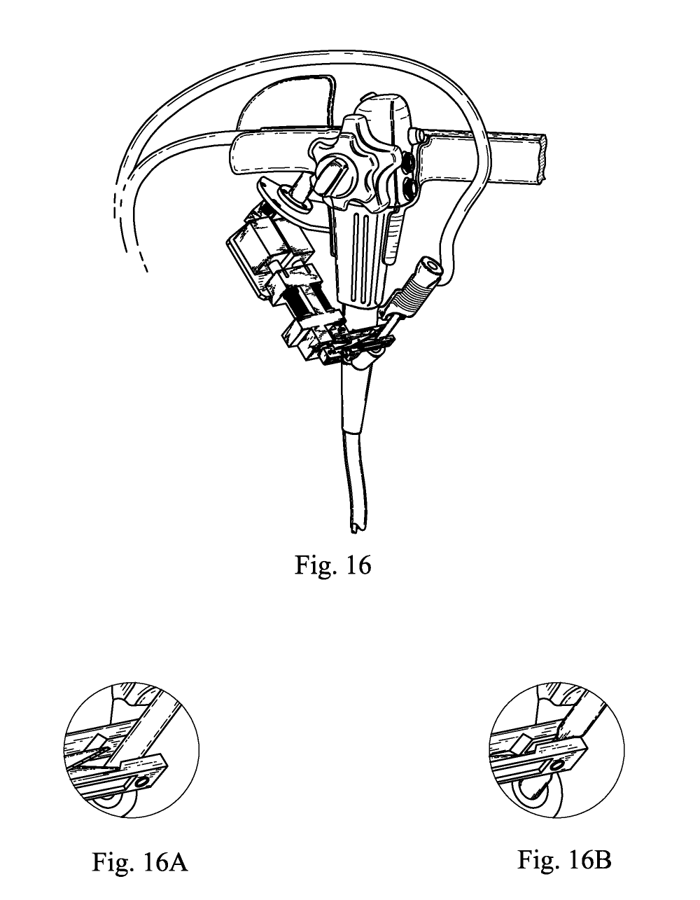

In FIGS. 14 and 16 is shown a situation when the working assembly is in the first working position and the heating jaws are forcibly removed from each other by the displacement solenoids. For the sake of simplicity only the exposed portion of the multilumen tubing is shown. This portion is placed between the heating jaws. Biasing members exert pressure on the heating jaws and on the portion of the multilumen tubing situated therebetween. At the same time the contact surfaces are heated by electrical power supplied thereto and heat the multilumen tubing. Since the multilumen tubing is made of a thermoplastic material, it yields and squeezes due to simultaneous influence of pressure and heat. In practice the heating surfaces are heated up to 230-25° C. After the sealing is completed, the displacement solenoids displace the traction rods to open the space between the jaws. The position solenoid 74 retracts its working pin from aperture 70 and the support arm is free to rotate together with the working assembly in the anticlockwise direction up to the second working position.

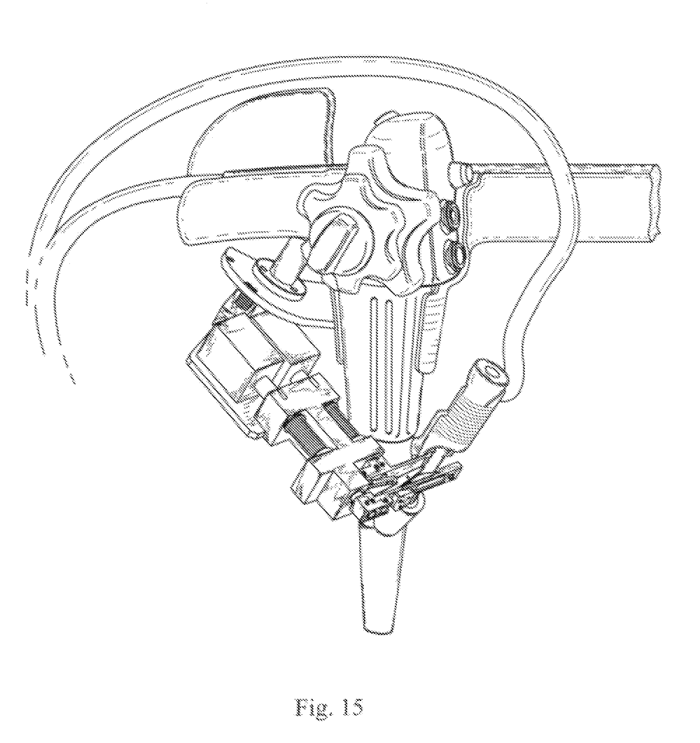

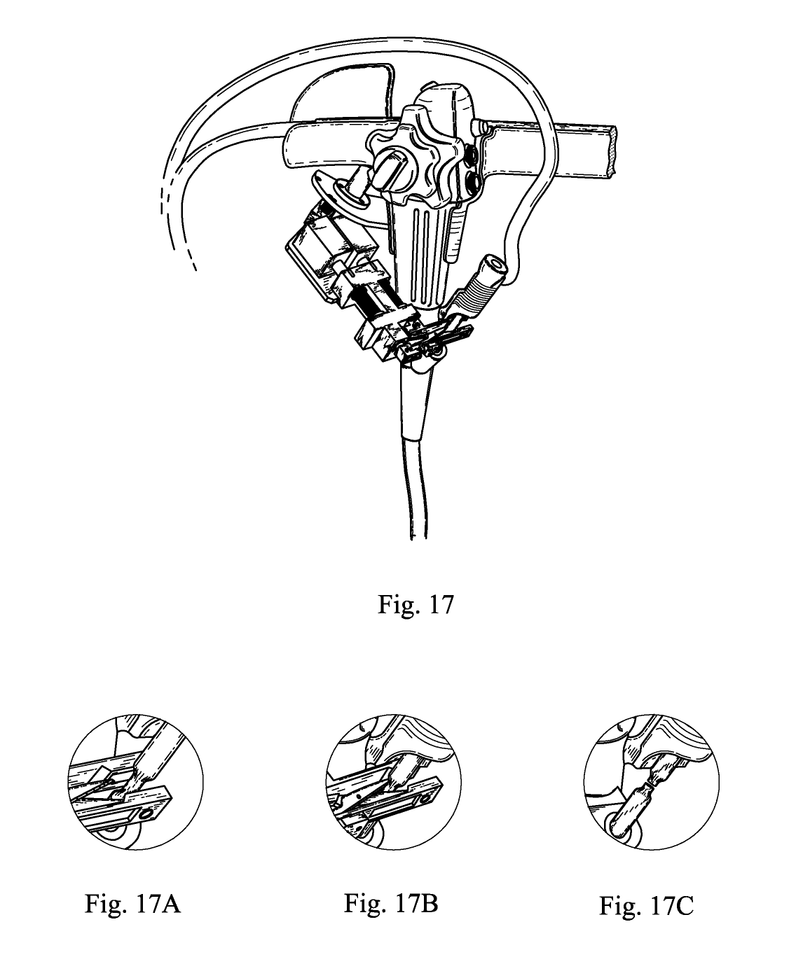

In FIGS. 15 and 17 is shown the situation when the working assembly is brought in the second working position. In this position the scissors approach the multilumen tubing. The displacement solenoids displace the scissors, such that cutting edges of the scissors cut the multilumen tubing. When the multilumen tubing is cut off, its opposite cut ends 150, 152 become heat-sealed as seen in FIG. 17c.

It can be readily appreciated that now, when the opposite ends 150, 152 of the multilumen tubing are heat-sealed, there is no possibility for the contamination accumulated during the endoscopic procedure to spread out from the proximal extremity of the multilumen tubing.

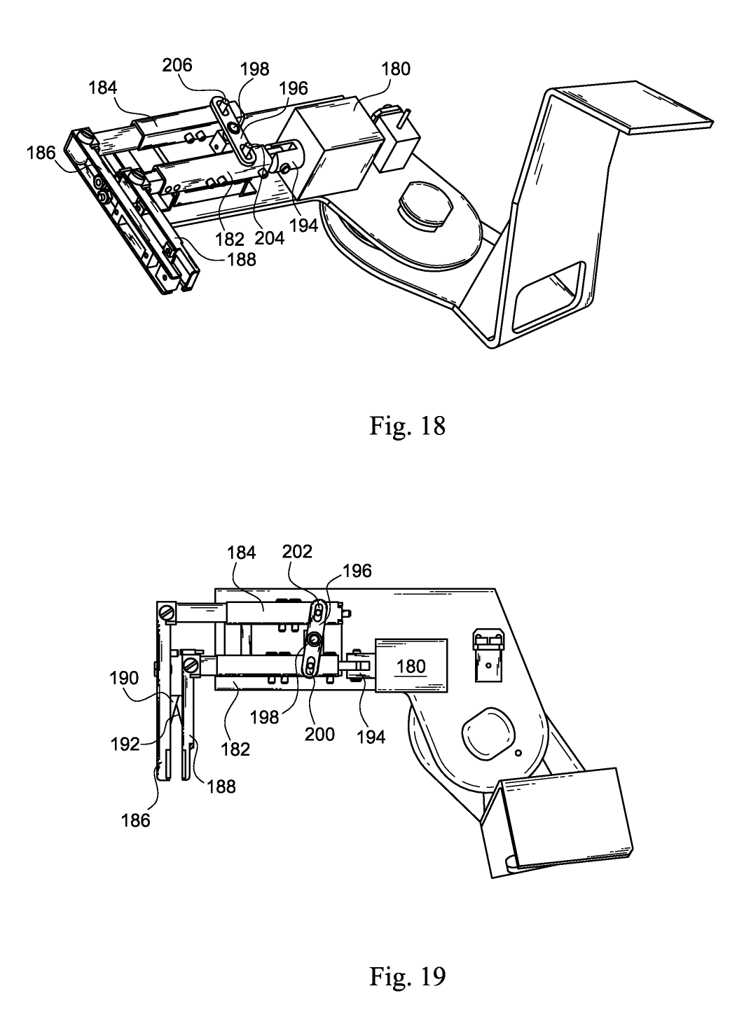

With reference to FIGS. 18, 19 a second embodiment of the working assembly is shown. In this embodiment the driving portion is provided with only one solenoid 180, which is capable to operate simultaneously two traction rods 182, 184. Each of the traction rods is connected to respective jaw 186, 188. In contrast to the previous embodiment the cutting action is carried out not by scissors but by two cutting blades 190, 192 provided at respective jaws. Only one of the traction rods, namely rod 182 is connected to a working pin 194 of the solenoid. It is not seen, but should be appreciated that, each of the traction rods is biased by respective biasing element in such a manner that rod 182 is urged to protract, while rod 184 is urged to retract. Thus, when the solenoid is not energized, the traction rods are biased in opposite directions and there is no space between the jaws. Between the traction rods there is provided a pivot plate 196, which can pivot about an axle 198. The opposite ends of the pivot plate are provided with slots 200, 202, while the traction rods are provided with respective pins 204, 206, which protrude into respective slots. By virtue of this provision, linear displacement of one of the rods is associated with pivoting of the pivot plate. The pivoting is translated in linear displacement of the second rod. The arrangement being such that when the solenoid is energized it retracts rod 182 and at the same time applies rotation moment on the end of the pivot plate connected to the rod 182 via pin 204. The rotation moment will be also applied to the opposite end of the pivot plate that is connected to the rod 184 via pin 206. The rotation moment will cause protraction of the rod 184. Simultaneous retraction of the rod 182 and protraction of the rod 184 will be associated with displacement of the jaws in opposite direction and with the opening the space therebetween. Now the exposed portion of the multilumen tubing can be placed between the jaws and sealed as it has been explained above in connection with the previous embodiment. After the sealing action is completed, the jaws are moved apart, the support arm with the working assembly is rotated up to the second working position. In this position the jaws are again brought together by the solenoid and cutting blades 190, 192 carry out the cutting action.

The invention is described in detail with reference to a particular embodiment, but it should be understood that various other modifications can be effected and still be within the spirit and scope of the invention.

We claim:

1. A device for cutting and sealing of a proximal, exposed portion of a multilumen tubing connected to an operating handle of an endoscopic apparatus when it is suspended on a hanger connected to a vertical post of a stand, said device comprising:
A working assembly and
A support assembly,
said working assembly having
i) a driving portion,
ii) a guiding portion and
iii) a working head with a first jaw and a second jaw juxtaposed to said first jaw, said first jaw and second jaw being relatively displaceable with respect to each other, said first and said second jaw being provided with respective first and second heating surfaces and with a source of energy for heating of at least one of the said first and the second surfaces, said first and second jaws deforming and sealing an exposed portion of the multilumen tubing upon heating of the at least one surface followed by relative displacement of the first and second jaws so as to bring the exposed portion in contact with the first and second surfaces and upon further relative displacement said first and second jaw subsequently cut the exposed portion;
wherein said device is mounted on the hanger for rotation of the working assembly and bringing the working head to the exposed portion of the multilumen tubing.

2. The device as defined in claim 1, wherein said working assembly is rotatable in a first direction and in an opposite second direction, said working assembly is rotatable about a rotation axis, which is not parallel to the vertical post of the stand, wherein said working assembly is forcibly rotatable in the first direction and is free to rotate in the second direction .

3. The device as defined in claim 2, wherein said support assembly is secured on a rear portion of the hanger.

4. The device as defined in claim 1, wherein said support assembly comprises a z-like bracket with a flat upper portion which embraces the hanger and with a flat lower portion which carries a shaft about which the working assembly is rotatable.

5. The device as defined in claim 4, wherein said support assembly further comprises a support arm, which carries the working assembly, said support arm is rotatable about the shaft.

6. The device as defined in claim 4, wherein said shaft is rigidly secured at the lower portion of the z-like bracket.

7. The device as defined in claim 4, wherein said upper portion is not parallel to the lower portion, wherein said upper portion is connected to the lower portion by an intermediate portion.

8. The device as defined in claim 4, wherein said bracket is provided with a means for limiting rotational movement of the support arm.

9. The device as defined in claim 4, wherein said bracket is provided with an adjustment mechanism for adjusting location of the upper portion of the bracket with respect to the hanger.

10. The device as defined in claim 4, wherein the working assembly is displaceable between a plurality of discrete working positions.

11. The device as defined in claim 10, wherein said working assembly is securable in a discrete working position and releasable therefrom, such that it can be brought to a further discrete working position and secured therein.

12. The device as defined in claim 11, wherein said lower portion of the bracket is provided with a plurality of apertures corresponding to the working positions and the working assembly is provided with a solenoid fitted with a pin, which upon energizing of the solenoid is protractable into and retractable from the said apertures such that the working assembly is respectively secured in the working position or is released therefrom.

13. The device as defined in claim 2, wherein said axis is inclined with respect to the post at a first inclination angle and at a second inclination angle

14. The device as defined in claim 1, wherein said driving portion comprises a first solenoid provided with a first rod and a second solenoid provided with a second rod, said first rod is operatively coupled with the first jaw and said second rod is operatively coupled with the second jaw, such that upon energizing of the solenoids said rods are protractable and retractable to relatively displace the jaws.

15. The device as defined in claim 14, wherein said guiding portion comprises a housing, accommodating therein a first biasing element, which is connected at a forward portion of the first rod and a second biasing element, which is connected at a rearward portion of the second rod, such that the first biasing element and the second biasing element urge the rods to be displaced in opposite directions.

16. The device as defined in claim 1, wherein at least one jaw is fitted with a cutting element.

17. The device as defined in claim 16 wherein both jaws are provided with the cutting element.

18. The device as defined in claim 17, wherein the cutting element is a scissors.

19. The device as defined in claim 1, wherein at least one thermocouple is provided for measuring temperature of at least one surfaces.

20. The device as defined in claim 19, wherein said device is provided with a mechanism for controlling the sealing time.
